(19) United States
(12) Patent Application Publication
(10) Pub. No.: US 2010/0287189 A1
(43) Pub. Date: Nov. 11, 2010

(54) ACCELERATION OF TAG PLACEMENT USING CUSTOM HARDWARE

(75) Inventor: Kent Allan Vander Velden, Perry, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignee: Pioneer Hi-Bred International, Inc.

(21) Appl. No.: 12/773,682

(22) Filed: May 4, 2010

Related U.S. Application Data
(60) Provisional application No. 61/175,669, filed on May 5, 2009.

(51) Int. Cl. G06F 17/30; A01H 1/00

(52) U.S. Cl. 707/769; 800/260; 707/E17.014

(57) ABSTRACT

A hardware device is configured to accelerate the process of determining the location of a query sequence (a tag) within a sequence library (such as a reference genome) using one or more comparison units having inputs for receiving the query sequence and a subsequence of the sequence library (a k-mer) and an output for reporting results where each comparison unit is capable of searching the tag in the sense and the antisense orientation against the sequence library in the sense and antisense orientation. Methods and systems are also provided.

### CROSS REFERENCE TO RELATED APPLICATIONS

The present application claims priority from U.S. Provisional Application No. 61/175,669, filed May 5, 2009, which is hereby incorporated herein in its entirety by reference.

### FIELD OF THE INVENTION

The present invention relates to computational biology. More specifically, the present invention relates to tag placement.

### BACKGROUND

With the relative wealth of genomic sequence available, biology is now turning to the use of the genomic sequences to accelerate discovery. Researchers use conventional sequencing techniques to obtain short subsequences of a target organism's DNA or RNA. The researchers then match these sequences back to the reference genome to identify features, such as single nucleotide polymorphisms (SNPs), to make observations about the organism's genetic makeup such as which alleles a given organism possesses, or to answer other related questions. Regardless of the desired end, the process is essentially the same. The researcher has one or more query sequences (also called tags) that they wish to search against a reference library (which may contain sequences from a number of species, one or more entire genomes or a subset thereof). The researcher's goal often is to place the tag within the sequence library.

Multiple approaches have been taken in an attempt to facilitate placement of the tag within the sequence library. Examples of these approaches include “brute force” methods, the Knuth-Morris-Pratt algorithm, and various hash and stored structure approaches. These approaches are notoriously inefficient (O(KR) time for the “brute force” approach and O(R) for Knuth-Morris-Pratt). By way of example, a comparison of 10⁶ query tags 32 nucleotides in length against a sequence library 3×10⁹ bases in length using a “brute force” approach on a 3 GHz quad-core processor would require approximately 73 years to complete if that processor could perform one comparison per clock cycle.

The chemical nature of DNA further complicates the problem. DNA consists of long polymers of simple units called polynucleotides. The asymmetric ends of DNA strands are referred to as the 5′ (five prime) and 3′ (three prime) ends, with the 5′ end being that with a terminal phosphate group and the 3′ end that with a terminal hydroxyl group. In living organisms, DNA does not usually exist as a single molecule, but instead as a tightly-associated pair of molecules. These two strands run in opposite directions to each other and in an arrangement termed anti-parallel. The backbone of the DNA strand is made from alternating phosphate and sugar residues. Attached to each sugar is one of four types of molecules called bases. Each type of base on one strand forms a bond with just one type of base on the other strand. This is called complementary base pairing. Here, purines form hydrogen bonds to pyrimidines, with adenine (A) bonding only to thymine (T), and cytosine (C) bonding only to guanine (G). A DNA sequence is called “sense” if its sequence is the same as that of a messenger RNA copy that is translated into protein. The sequence on the opposite strand is called the “antisense” sequence.

For many applications an effective search requires comparing the query sequence in the sense orientation and the sequence library in the sense orientation, the query sequence in the antisense orientation and the sequence library in the sense orientation, the query sequence in the sense orientation and the sequence library in the antisense orientation, and the query sequence in the antisense orientation and the sequence library in the antisense orientation. Performing all four comparisons effectively quadruples search time in the above example.

When using sorted structure approaches, an exact match for a single tag can be performed in O(log(R)) time (instead of O(R) time). To allow mismatches, each tag is expanded into a set of tags, H, which are within the maximum allowed Hamming distance. With this approach, as the maximum number of mismatches, M, grows, H grows much faster. An upper bound is

$$\vert H \vert \leq {\sum }_{m = 0}^{M} ( \begin{matrix}K \\ m\end{matrix} ) \times {3}^{m}$$

which shows the size of H grows factorially because of the combinatorial term. As a result, this method is only practical up to only a few mismatches. Additionally, rotating media is not suitable for access patterns necessary for this method.

The CPU methods are parallel at several different levels, but parallelization on a general purpose CPU only reduces the runtime by a linear term, the number of parallel CPUs.

Thus runtime is only marginally improved. What is needed is a method, apparatus, or system that accelerates tag placement within a sequence library.

### SUMMARY OF VARIOUS EMBODIMENTS

The process of finding where a query sequence (a tag) is located on a sequence library (such as a reference genome) is accelerated using a device. This acceleration is accomplished through the use of one or more comparison units capable of searching the tag in the sense and the antisense orientation against the sequence library in the sense and antisense orientation. The plurality of comparison units may be connected in parallel. The comparison units have inputs for receiving the query sequence and a subsequence of the sequence library and an output for reporting results.

The device may be operatively connected to a host system, which may be comprised of a general purpose computer. When so connected, functionality may be split between the host system and the device.

The device may be used to facilitate a number of common techniques in genetics and the biological sciences. In an example of one such use the device may be used in a plant breeding program to identify genomic regions that are linked with desired phenotypic traits. These genomic regions may then be used to improve the phenotype of other plant lines.

### BRIEF DESCRIPTION OF THE DRAWINGS

FIG. 1 is a diagram showing the operation of the apparatus for identifying the location of tags within the sequence library.

FIG. 2 is a circuit diagram showing the driver unit 80 in relationship to the communication layer 70.

FIG. 3 is a circuit diagram showing the driver unit 80 which contains the comparison driver 180.

FIG. 4 is a circuit diagram of the comparison driver 180 showing the comparison units 220 connected in parallel.

FIG. 5 is a diagram showing scatter and gather to and from the replicated comparison units 220 connected in parallel.

FIG. 6 is a circuit diagram showing the composition of a comparison unit 220.

FIG. 7 is a graph depicting the measured runtime of a hardware-based and software-only version of the apparatus.

FIG. 8 is a graph depicting the measured speedup of the hardware-based version over the software-only version of the apparatus.

FIG. 9 is a diagram showing a host system operatively connected to a custom hardware device comprised of a plurality of comparison units configured to identify the location of a query sequence within a sequence library.

FIG. 10 is a diagram showing a plant breeding program that uses a custom hardware device to facilitate the identification of genomic features correlated with genes that result in a desirable phenotype in a plant line.

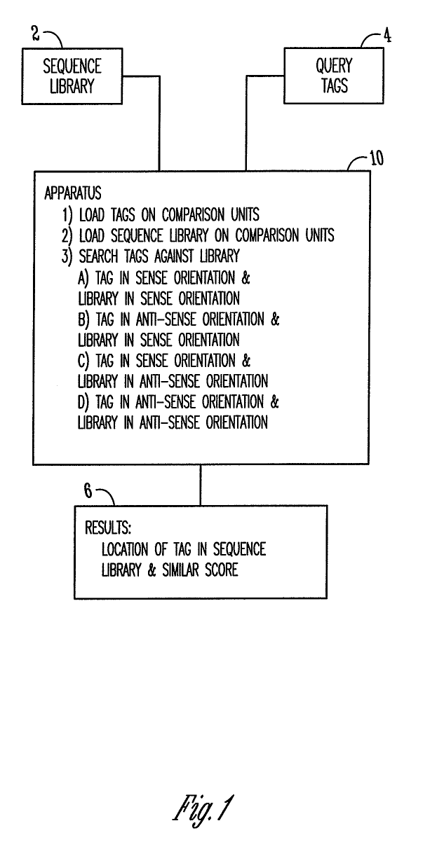

### DETAILED DESCRIPTION

In some aspects, tag placement may be thought of as a string search problem. The tag is a string of length K and the genome is a much larger string of length R. A “brute force” text search method operates in O(KR) time, but methods exploiting mismatches between the query and the reference operate in O(R) time (e.g. Knuth-Morris-Pratt). Unfortunately, these methods are only suitable when performing exact matches. Effective tag placement requires that some amount of mismatch between the tag and the reference is allowed to compensate for sequencing error, evolutionary drift, and other sources of mismatches. The accommodation of mismatches significantly complicates tag placement.

A “brute force” method of performing tag placement is shown below. The idea is to measure the Hamming distance between each tag and the K-length substring starting at each position of the genome. If the Hamming distance is less than or equal to a maximum threshold, the position is recorded as a tag match.

```
for i = 1 to genome.length
  for j = 1 to tags.count
    hd = 0
    for k = 1 to tags[j].length
      if genome[i] != tags[j][i]
      hd = hd + 1
      end if
    end
    if hd <=max_hd
      record match at position i
    end if
  end
end
```

To understand the difficulty of this method, consider a genome of length R=3×10⁹ (three giga-bases) and N=10⁶ tags (a reasonable number for a Solexa machine) where each tag is K=32 in length. The total number of comparisons necessary for the method above. is then RxNxKx2=192×10¹⁵. If a processor could perform one comparison per clock cycle, even a 3 GHz quad-core processor would require 73 years to complete.

Hashing and sorted structure approaches trade increased storage space requirements to gain increased speed. Allowing additional storage to be used, the genome can be preprocessed into tags of length K and stored in a sorted structure. Performing exact match for a single tag against this sorted structure can be performed in O(log(R)) time (instead of O(R) time). To allow mismatches, each tag is expanded into a set of tags, H, which are within the maximum allowed Hamming distance. Each tag in H then forms another query. This leads to the algorithm below.

Stage 1:
For every k-mer in genome, store k-mer and its position in a sorted structure

Stage 2:
For every tag, query sorted structure and report positions

Stage 3:
For every tag, construct the set H of strings that are 1, 2, . . . maxHD Hamming distance from the tag

Stage 4:
For every tag in H, query sorted structure and report positions

The improved algorithm has better theoretic runtime performance over the previous version as long as the size of H does not exceed R/log(R). Mismatches present difficulties because as the maximum number of mismatches, M, grows, H grows much faster. An upper bound is

$$\vert H \vert \leq {\sum }_{m = 0}^{M} ( \begin{matrix}K \\ m\end{matrix} ) \times {3}^{m}$$

which shows the size of H grows factorially because of the combinatorial term. As a result, this method is only theoretically practical up to a few mismatches.

Additionally, the second method does not scale as well as expected due to an additional limitation caused by the demands of the sorted structure holding the genome. Rotating media is not suitable for access patterns necessary for this method. Improvements can be made to address this limitation, but this does not improve the theoretic bounds.

The first CPU method discussed is slow because of several large linear multiplicative terms. The second CPU method is faster because it converts the largest linear term to a logarithmic term, but at the expense of introducing a factorial term. With advances in sequencing technology, tag sizes are growing, and longer tag sizes require additional mismatch allowance which is the main source of computational complexity.

Based on experience with bottlenecks in software design, a new design was formulated using custom hardware. A sequence library 2 and query tags 4 are supplied to an apparatus 10 comprising a plurality of comparison units configured to search the query tag in the sense orientation against the sequence library in the sense orientation, the query tag in the sense orientation against the sequence library in the antisense orientation, the tag in the antisense orientation against the sequence library in the sense orientation, and the tag in the antisense orientation against the sequence library in the antisense orientation. The apparatus 10 then outputs results 6 comprised of the location of the tag in the sequence library and a similarity score.

When combined with a software component, the software component may execute on host system 390 comprised of a general purpose computer. The host system 390 may perform I/O operations such as loading tags, loading the genome, interrupting results, and saving results.

The custom hardware 10 is responsible for identifying the location of the tag within the sequence library (which may be comprised of one or more sequences that may or may not all be from the genome of a target organism). The custom hardware 10 may be operatively connected to the host system 390 using a number of protocols including CPU buses (e.g. front side bus, HyperTransport, and QuickPath), peripheral component interconnect (PCI), peripheral component interconnect express (PCIe), high speed serial connections, universal serial bus (USB), and AT Attachment (e.g. IDE, EIDE, SATA). In such a configuration the host system 390 may communicate such information as the tags, the sequence library, and configuration data (including the sequence orientations to compare, the maximum Hamming distance, and other parameters) to the custom hardware 10.

Because the CPU of the host system 390 acts independently of the custom hardware 3, the work performed by each may overlap. To take advantage of this, and to minimize communication, the system may be designed such that the results returned from the custom hardware only indicate if at least one tag matched at a given position. Under this approach, the custom hardware results do not explicitly indicate which tags match the sequence library and do not indicate their Hamming distances. Instead, while the custom hardware 10 processes the next frame, the CPU examines the results from the previous frame, received from the custom hardware, and disambiguates precisely which tags matched. The disambiguation may be done using described CPU methods. Because matches are infrequent, disambiguation on the CPU does not become a bottleneck.

Under this approach, the software streams the data frames to the custom hardware and indicates if a match was detected by the custom hardware. Of course, the relative roles of the hardware and the software may be adjusted in various ways to facilitate providing the query tags and the sequence library to the comparison units in the apparatus.

The logic required for the custom hardware may be implemented by any means known in the art such as custom designed silicon chip including full custom design and application specific integrated chips (ASIC), or using a reconfigurable semiconductor device including a field programmable gate arrays (FPGA) and complex programmable logic devices (CPLD), or other types of devices or reconfigurable devices used for hardware implementations.

Full custom design is where an integrated circuit is designed by specifying the layout of each individual transistor in the circuit and the connections between the transistors. Although expensive, this approach allows for the creation of custom built-to-order semiconductor chips.

An ASIC is a type of integrated circuit, often referred to as “gate-array” or “standard-cell” products, developed and designed to satisfy a specific application requirement. Using basic assemblies of logic elements called standard cells, functional blocks with known electrical characteristics, such as propagation delay, capacitance and inductance, may be created and used to implement customized hardware solutions. The result is a largely custom chip at a reduced expense from a full custom design chip.

FPGAs and CPLDs are examples of reconfigurable semiconductor devices. Both FPGAs and CPLDs may be thought of as a matrix of logic elements. Specialized tools take designs specified in hardware description languages (HDL) and synthesize a binary stream that configures the FPGA to implement the hardware design. Reconfigurable semiconductor devices are a useful tool for hardware designers allowing designers to test a physical design without the great expense of fabricating an actual chip.

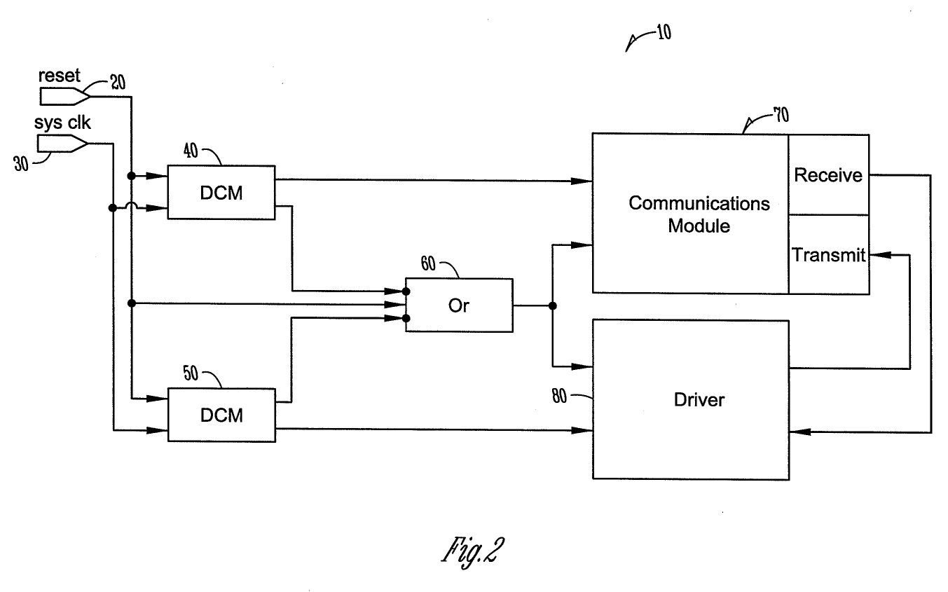

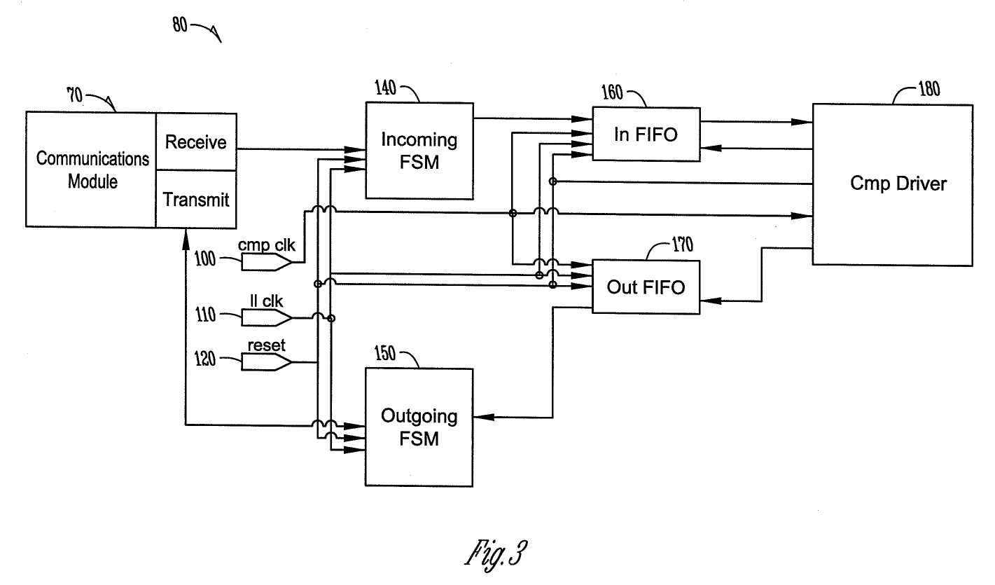

It is useful to describe the custom hardware by way of an example. Looking at the example hierarchically, at the highest level the FPGA 10, in addition to being connected to a host system via Ethernet, receives two inputs (reset 20 and sys clk 30) from the host system. These inputs 20, 30 are electrically connected to two digital clock managers (DCM) 40, 50. The DCM modules 40, 50 and the reset 20 are connected to a communications module (e.g. a media access controller (MAC)) 70 and driver 80. Possible communication protocols for the communications module includes, but is not limited to, CPU buses (e.g. front side bus, HyperTransport, and QuickPath), peripheral component interconnect (PCI), peripheral component interconnect express (PCIe), high speed serial connections, universal serial bus (USB), and AT Attachment (e.g. IDE, EIDE, SATA). The driver module 80 (FIG. 2 and FIG. 3) is comprised of a plurality of comparison units 220 and is operatively connected to the communication module 70.

The driver module 10 helps to bridge clock domains and simplifies the interface to the communication layer by removing and adding communication headers. The driver module 10 contains two finite state machines (FSM) 140, 150 running in parallel. An incoming FSM 140 is electrically connected to the transmitting portion of the communications module 70 and the In FIFO module 160. The In FIFO 160 module is electrically connected to and buffers data going into the comparison driver module 180. The comparison driver module 180 is electrically connected to the Out FIFO module 170. Data leaving the comparison driver module 180 is buffered by the Out FIFO module 170. The Outgoing FSM 150 is electrically connected to the Out FIFO module 170 and the receiving portion of the communications module 70. The Outgoing FSM 150 pulls data from the Out FIFO module 170 and sends the data to the communications module 70.

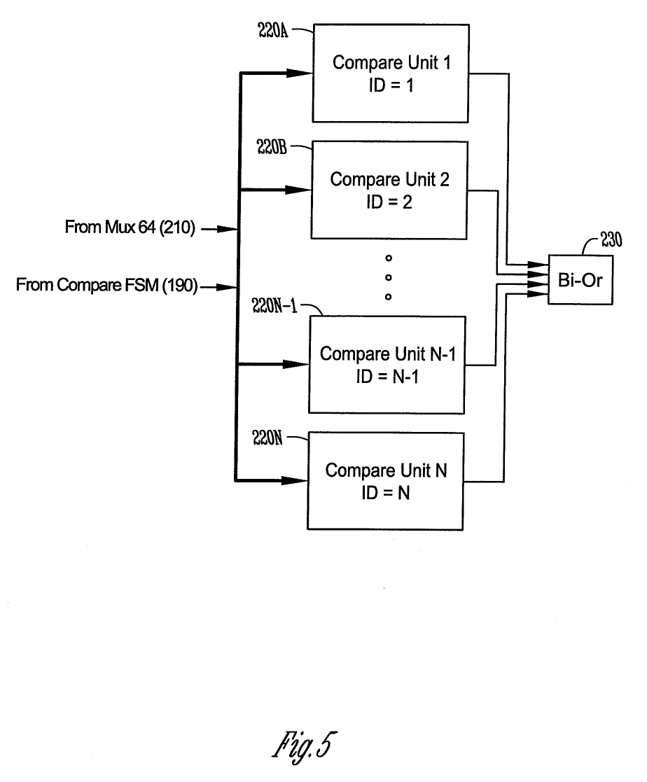

The comparison units driver 180 is comprised of a Compare FSM 190 which is electrically connected to the In FIFO module 160 and the Out FIFO module 170. The Compare FSM 190 is also electrically connected to a Shift Unit 200 which is in turn connected to a 64-bit multiplexer (Mux 64) 210. The Shift Unit 200 takes the input sequence library stream and produces a subsequence of the sequence library (such as a 32 nucleotide substring (“32-mer”)) in overlapping segments for each clock cycle (each nucleotide may be encoded as two bits). Of course, the Shift Unit 200 may be configured to produce non-overlapping segments as well. The shift may be accomplished by shifting in two new bits (one nucleotide) from the input stream while shifting out the oldest two bits. External to the Shift Unit 200, only the 64-bit value is observed. The multiplexer 210 switches the input source of the comparison units from the Shift Unit 200 to one coming directly from the Compare FSM 190. This loads the initial tags without the lag of sending them through the Shift Unit 200. The Multiplexer 210 is electrically connected to one or more comparison units 220(A-N) and provides the comparison units 220(A-N) with the query sequences. The Compare FSM 190 is also connected to the comparison units 220(A-N) and controls the comparison units 220(A-N) operation. Tag and hamming distance loading decisions are controlled by the ID unit 370, the Compare9 unit 360, and two And gates 350, 380. The Compare9 unit 360 returns a logical 1 if all corresponding bits in two nine-bit values are equal, else 0 is returned. Of course, the logic may be reversed to achieve similar results. This logic drives the two single 2×1 multiplexers 340, 236. This ID 370+Compare9 360+And 350, 380+multiplexer 340, 236 logic allows the Tag to be loaded from the sequence library stream or for the same Tag to be reused by the comparison unit 220(A-N). Results from the comparison units 220(A-N) are reported via an electrical connection to a Bi-Or module 230 which aggregates the results and reports the aggregated results via an electrical connection to the Compare FSM 190. The Compare FSM reports results via an electrical connection to the Out FIFO module 170. An alternative view of this scatter and gather mechanism can be seen in FIG. 5.

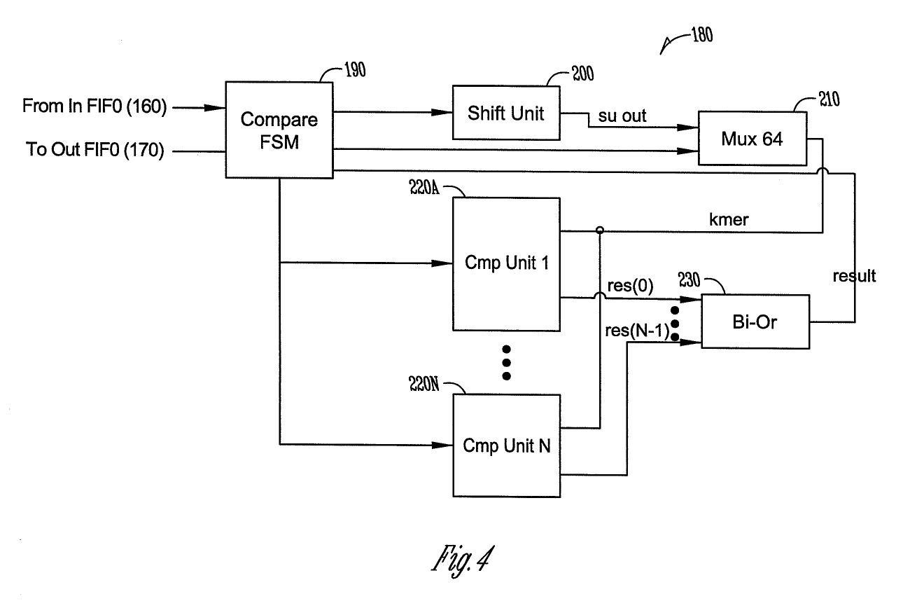

Replication of the comparison unit 220 provides parallelism (FIG. 5), and driving the comparison units 220(A-N) is a FSM 190 controlling all the signal lines (FIG. 4). Additional hardware for compressing results and providing the genome substrings in overlapping tag-length length K substrings may be provided (also called a “K-mer”). The FSM 190 is responsible for loading each comparison unit 220 with its required information such as the tag, the maximum Hamming distance, and which directions the tag and genome should be examined. The FSM 190 also controls signals which are propagated back to the previous level to shuffle values in and out of the FIFOs 160, 170.

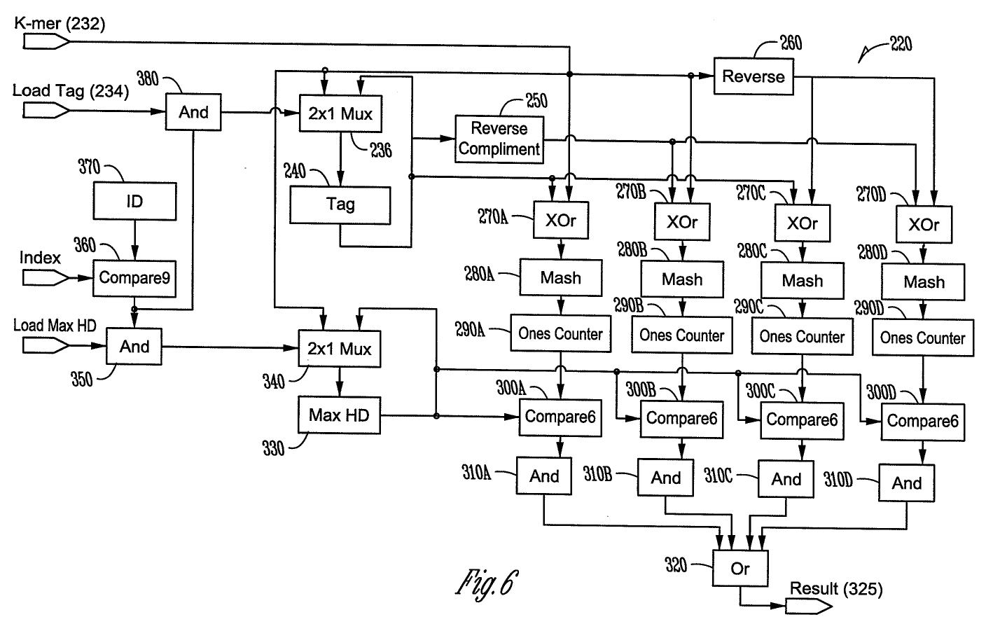

The comparison unit 220 is where a substring from the sequence library is compared against a tag (FIG. 6). The comparison unit has an input for the query sequence 232, an input for a subset of the sequence library 234, and an output for reporting the result 325. Looking in closer detail, two 2×1 Multiplexers 236, 340 are electrically connected to the FSM 190 and two registers 240, 330 and control the flow of data into the registers 240, 330. The two registers 240, 330 hold the tag 240 and the maximum allowed Hamming distance 330. Of course the comparison unit 220 may be configured to store a plurality of tags and subsequences of the sequence library to further optimize the comparison unit 220. Tag reversal, including generation of the reverse complement representation, is implemented using hardware permutation that translates to routing hardware 250, 260. Four parallel paths through the comparison unit examine all orientations of the tag and genome substring at once. This is achieved by electrically connecting the tag register 240, the reverse complement module 250, and the reverse module 260 to the comparison logic 270(A-D), 280(A-D), 290(A-D), 300(A-D) and 310(A-D) such that one path receives the query sequence in the sense orientation and the sequence library in the sense orientation, another path receives the query sequence in the antisense orientation and the sequence library in the sense orientation, another path receives the query sequence in the sense orientation and the sequence library in the antisense orientation, and the final path the query sequence in the antisense orientation and the sequence library in the antisense orientation. If fewer than four orientations are required, one or more of the parallel paths may be disabled or discarded from the design.

Each path compares the tag to the subsequence using an Exclusive Or (XOr) 270 which identifies whether there are any mismatches. The result is then passed via an electrical connection to a “Mash” module 280 which performs an “Or” between each even bit and its neighboring odd bit. After applying the “Mash” logic, the number of ones represents the number of mismatches. As such the hamming distance can be calculated by summing the ones. The summation of the bits may be accomplished using any known technique such as a binary tree of adders approach. A Ones Counter 290 is electrically connected to the “Mash” module 280 and performs the summation. The Compare6 300 unit is electrically connected to the Ones Counter 290 and the register containing the maximum hamming distance 330. The Compare6 unit 300 returns a single bit if the actual Hamming distance is less than or equal to Max HD. The And 310 module is electrically connected to the Compare6 module 300 and may be used to mask the results from non-desired orientations. An Or module 320 is electrically connected to the And modules 310(A-D) and may be used to reduce the result to a single bit indicating whether the tag matched the subsequence from the sequence library. This result is then returned to the BiOr module 230.

The multiple paths present a marked improvement over traditional approaches using a standard CPU. Under the traditional approaches, reversal requires as many steps as the length of the tag or O(N) time. Using parallel paths results in no delay as the reversal is done via connections of wires resulting in no computational cost. Additionally, the hardware design may be configured to count the ones using a tree structure of adders resulting in O(log(N)) time instead of O(N) required by a CPU. Further optimization may be accomplished through binary encoding of the nucleotides (such as A=00, C=01, G=10, T=11) such that complementation may be performed using bitwise complement avoiding the need for lookup tables or conditionals.

The driver module 160 may handle incoming and outgoing data in parallel. The comparison unit 220 may be replicated until the space within the custom hardware solution is exhausted. The comparison unit 220 itself may be designed to have four parallel pipelines, measure the Hamming distance in parallel, and perform the otherwise linear operation of reversal without any computational cost. No analogous operation to reversal exists on a CPU.

A further benefit of this design is considerable power savings. For example, an FPGA board, with the required host computer, power consumption is approximately 330 W. Given that the design is 390-fold faster than a single computer, an equivalent number of computers would be required to complete the process in the same time requiring many kWs of power. Because of the cost of assembling and supporting the 390 computers, one would likely use fewer computers and allow more time for the analysis to complete. This approach would still consume significantly more power than the custom hardware implementation.

The apparatus may be configured to accept and search any given size of tag (e.g. tags 16, 32, 64 nucleotides in length) by varying the register sizes and making other appropriate adjustments to the circuits. Additional flexibility may be achieved by configuring the apparatus to handle a given upper tag size limit and using masking to accommodate tags of smaller lengths.

Tag storage and comparison may be done using different logical approaches. For example, the tags may be loaded from a configuration packet and remain unchanged as the sequence library is streamed to the comparison units 220. In another alternative, multiple tags are stored in the comparison unit 220 and each tag is compared to the sequence library with a single streaming.

Additionally, even greater speedup may be achieved by optimizing the apparatus. One approach is by increasing the clock rate of the apparatus. Increases in clock rate may be achieved by pipelining through the use of the unused registers in the FPGA as additional memory. Additionally, use of timing constraints also enables increases in the clock rate.

### Example 1

For purposes of comparison a software only version and an FGPA based implementation were created. The software only version was run on an Intel q6600 2.4 GHz quad core processor and 8 GB of 800 MHz DDR2 memory. The FPGA based implementation was run on an ML507 evaluation board from Xilinx with Virtex-5 FX70T FPGA. The host computer was a 3.0 GHz Pentium D is connected to the board by gigabit Ethernet. In addition to the Gb Ethernet the Xilinx ML507 offers connectivity through PCIe, high speed serial connections, DVI/VGA, USB, SATA, PS/2, various audio connections, and others. SRAM, DDR, and CF memories are also available. Of course, the apparatus may be implemented on any size of FPGA board. The use of larger FPGA boards allows for even greater speedup through the use of additional comparison units 220.

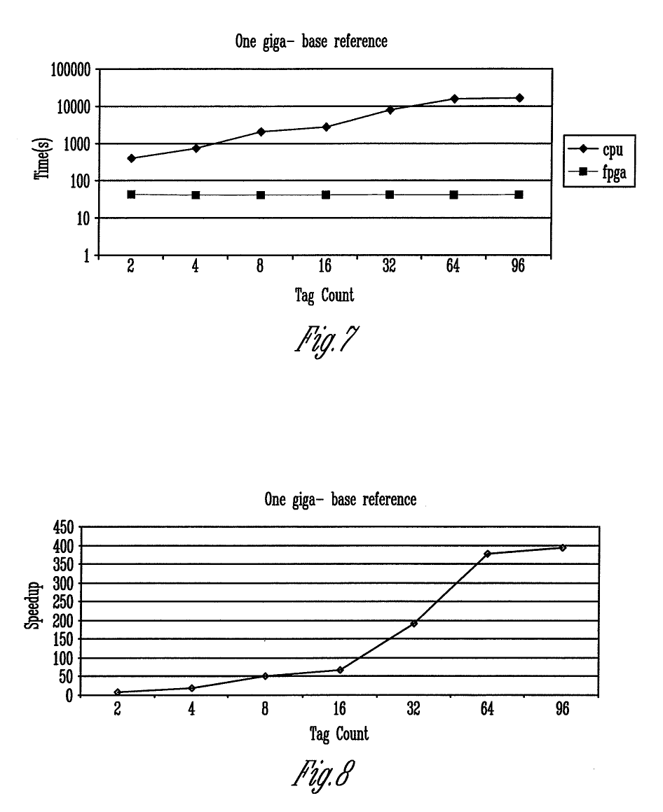

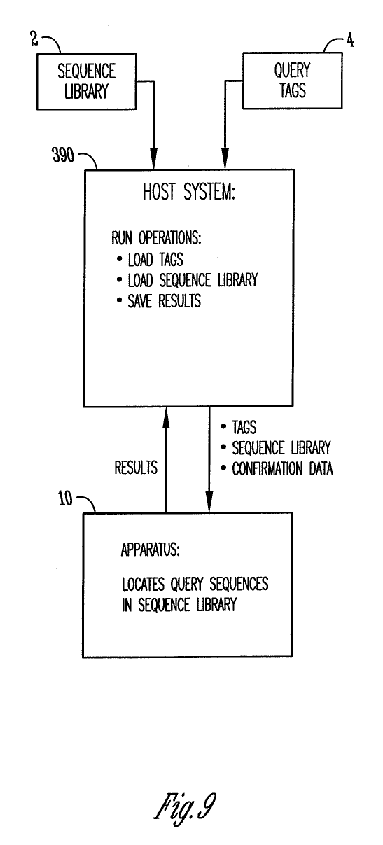

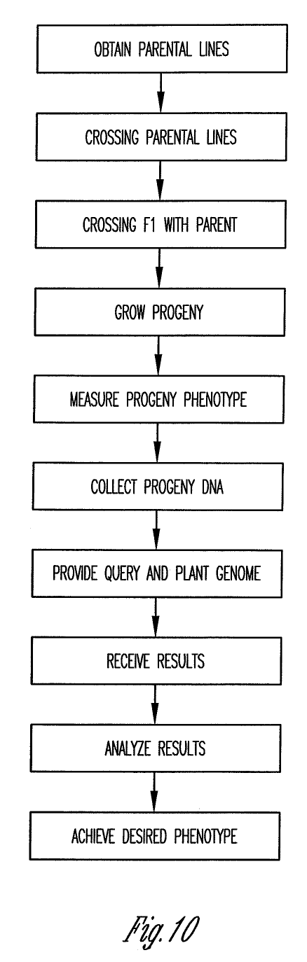

A one giga-base sequence library was used with query tags 32 bases in length, and 2, 4, 8, 16, 32, 64, and 96 tags were compared against the sequence library in all four possible orientations. FIG. 7 shows the relative times for the software only (cpu) and the fpga approaches (fpga). FIG. 8 shows the relative speedup observed by using the FPGA. As seen in FIG. 8 a 390-fold increase in performance over the software version was observed when 96 query tags were compared with the sequence library. This improvement was observed regardless of the number of mismatches. These results are particularly significant given that the FPGA design runs at 25 MHz and the CPU design runs at 2.4 GHz. Despite this large disparity in clock speeds, the FPGA design is two orders of magnitude faster.

### Example 2

The apparatus may be used as part of a plant breeding program. Many plant traits of economic importance are polygenic traits, meaning that the traits are controlled by multiple genes at more than one location in the plant's genome. Significant improvements in key plant traits may be accomplished by controlling the alleles a given plant line possesses at these genes. A goal of many plant breeding programs is to develop plant lines with increased numbers of desirable alleles at the genes controlling these traits. A difficulty has been determining the genetic makeup of the plant line.

Rather than expending the considerable time and resources of sequencing the entire plant genome, researchers have instead focused on finding readily identifiable features within the plant genome which are closely linked to the desired alleles. Features frequently used include restriction fragment length polymorphisms (RFLP), single nucleotide polymorphisms (SNP), Single Sequence Repeats (SSRs), Target Region Amplification Polymorphisms (TRAPs). Focusing particularly on SNPs, a SNP is a single nucleotide variation within the genomic sequence for a given species which may fall anywhere within the genome including within coding portions of the genome. For any given plant species, genomic maps identifying the location of these traits throughout the genome are publically and privately available. The process of statistically studying the alleles which occur in a particular gene and the resultant plant traits is called QTL mapping.

Typically, a researcher studying a trait will conduct a backcross experiment. In the experiment, two inbred parental plant lines 400 differing in a trait of interest will be crossed to form a first filial generation 410. This first filial generation is then crossed with one of the inbred parental lines 420. The progeny are grown 430 and the phenotype of the resulting progeny is measured 440. In addition to the phenotype, genomic DNA is collected from the progeny and the progeny are genotyped at markers spaced relatively evenly throughout the genome 450. Statistical analysis, such as analysis of variance (ANOVA), is then used to determine which markers are closely associated with the desired trait.

The apparatus may be used to facilitate the process of genotyping the progeny. In such an approach, genomic DNA from the progeny plants is sequenced using a high throughput sequencer such as an Illumina Genome Analyzer. The sequencer may be configured to produce a large number of short sequences (e.g. 36 nucleotides) that then become the query sequences. These query sequences and a reference genome is provided to the apparatus 460. The apparatus solution is then used to match these sequences the reference genome for the plant of interest 470. Using this approach, it is then possible to identify the SNP alleles present in the progeny plant. Using this data, the researcher can then apply ANOVA to determine which SNPs are most closely correlated (linked) with desirable phenotypic measurements in the traits of interest 480. Once the appropriate SNPs have been identified, a plant breeder may then use standard plant breeding techniques, such as marker assisted selection, to introduce the gene of interest into other plant lines in order to achieve a desired phenotype in those plant lines 490. Thus plant breeding may be significantly improved using the described apparatus.

A hardware device for acceleration of tag placement has been disclosed. The hardware device may be implemented on a custom-designed semiconductor chip such as a full custom design chip or an ASIC. Additionally, the hardware device may be implemented using a reconfigurable semiconductor device such as an FPGA or a CPLD. Optionally, a host system may be connected to the hardware device to facilitate the tag placement performed by the hardware device. Optionally, the host system may perform some of the I/O operations such as loading tags, loading the genome, interrupting results, and saving results. The host system may also perform disambiguation of results from the hardware device reducing communication between the custom hardware and the host system. The custom hardware may be configured with any number of comparison units that may be connected in parallel to provide even greater reductions in search time. Further optimization may be accomplished by employing such techniques as binary encoding of nucleotides, increased pipelining, and use of timing constraints.

Throughout the specification examples have been used to illustrate the present invention. It is to be understood that the present invention contemplates numerous variations, modifications, and alternatives. As such, the scope of the claims should not be limited by the various examples provided herein.

What is claimed is:

1. An apparatus for locating a query sequence in a sequence library, the apparatus comprising:
a plurality of comparison units operatively connected in parallel wherein each of the plurality of comparison units are configured for comparing (a) the query sequence in the sense orientation and the sequence library in the sense orientation, (b) the query sequence in the antisense orientation and the sequence library in the sense orientation, (c) the query sequence in the sense orientation and the sequence library in the antisense orientation, and (d) the query sequence in the antisense orientation and the sequence library in the antisense orientation;
each of the plurality of comparison units having an input for receiving the query sequence; each of the plurality of comparison units having an input for receiving a subsequence of the sequence library; and
each of the plurality of comparison units having an output for reporting results.

2. The apparatus of claim 1 wherein the plurality of comparison units is implemented in a custom designed silicon chip.

3. The apparatus of claim 2 wherein the plurality of comparison units is implemented in an application specific integrated chip.

4. The apparatus of claim 1 wherein the plurality of comparison units is implemented in a reconfigurable semiconductor device.

5. The apparatus of claim 4 wherein the reconfigurable semiconductor device is a field programmable gate array.

6. The apparatus of claim 1 further comprising a communications module operatively connected to the plurality of comparison units.

7. The apparatus of claim 1 wherein each of the plurality of comparison units is further configured for storing a plurality of additional tags.

8. The apparatus of claim 1 wherein the query sequence is comprised of no more than 32 nucleotides of the sequence library.

9. The apparatus of claim 1 wherein the sequence library is comprised of more than one sequence.

10. The apparatus of claim 1 wherein the sequence library is comprised of the genome of a target organism.

11. The apparatus of claim 1 wherein the apparatus is configured to encode the query sequence and the subsequence of the sequence library such that complementation may be performed using bitwise complement.

12. The apparatus of claim 11 wherein the apparatus is configured to encode the query sequence and the subsequence of the sequence library using a binary encoding method where A=00, T=11, C=10, and G=01.

13. A method for locating a query sequence in a sequence library, the method comprising:
providing a hardware device comprising a plurality of comparison units configured to locate the query sequence within the sequence library;
providing at least one query to the hardware device;
providing a plurality of subsequences of the sequence library to the hardware device; and receiving results from the hardware device indicative of the location of the query sequence within the provided subsequences of the sequence library.

14. The method of claim 13 wherein the plurality of comparison units is implemented in a custom designed silicon chip.

15. The method of claim 14 wherein the plurality of comparison units is implemented in an application specific integrated chip.

16. The method of claim 13 wherein the plurality of comparison units is implemented in a reconfigurable semiconductor device.

17. The method of claim 16 wherein the reconfigurable semiconductor device is a field programmable gate array.

18. The method of claim 13 further comprising a communications module operatively connected to the plurality of comparison units.

19. The method of claim 13 wherein each of the plurality of comparison units is further configured for storing a plurality of additional tags.

20. The method of claim 13 wherein the query sequence is comprised of no more than 32 nucleotides of the sequence library.

21. The method of claim 13 wherein the sequence library is comprised of more than one sequence.

22. The method of claim 13 wherein the sequence library is comprised of the genome of a target organism.

23. The method of claim 13 wherein the apparatus is configured to encode the query sequence and the subsequence of the sequence library such that complementation may be performed using bitwise complement.

24. The method of claim 23 wherein the apparatus is configured to encode the query sequence and the subsequence of the sequence library using a binary encoding method where A=00, T=11, C=10, and G=01.

25. An apparatus for locating a query sequence in a sequence library, the apparatus comprising:
a reconfigurable semiconductor device configured to include a plurality of comparison units wherein each comparison unit is configured for comparing (a) the query sequence in the sense orientation and the sequence library in the sense orientation, (b) the query sequence in the antisense orientation and the sequence library in the sense orientation, (c) the query sequence in the sense orientation and the sequence library in the antisense orientation, and (d) the query sequence in the antisense orientation and the sequence library in the antisense orientation.

26. The apparatus of claim 25 wherein the reconfigurable semiconductor device is a field programmable gate array.

27. The apparatus of claim 25 further comprising a communications module operatively connected to the plurality of comparison units.

28. The apparatus of claim 25 wherein each of the plurality of comparison units is further configured for storing a plurality of additional tags.

29. The apparatus of claim 25 wherein the query sequence is comprised of no more than 32 nucleotides of the sequence library.

30. The apparatus of claim 25 wherein the sequence library is comprised of more than one sequence.

31. The apparatus of claim 25 wherein the sequence library is comprised of the genome of a target organism.

32. The apparatus of claim 25 wherein the apparatus is configured to encode the query sequence and the subsequence of the sequence library such that complementation may be performed using bitwise complement.

33. The apparatus of claim 32 wherein the apparatus is configured to encode the query sequence and the subsequence of the sequence library using a binary encoding method where A=00, T=11, C=10, and G=01.

34. A method for locating a query sequence in a sequence library, the method comprising:
providing a reconfigurable semiconductor device configured to locate the query sequence within the sequence library using a plurality of comparison units;
providing at least one query sequence to the reconfigurable semiconductor device; providing a plurality of subsequences of the sequence library to the hardware device; receiving results from the hardware device indicative of the location of the query sequence within the provided subsequences of the sequence library.

35. The method of claim 34 wherein the reconfigurable semiconductor device is a field programmable gate array.

36. The method of claim 34 further comprising a communications module operatively connected to the plurality of comparison units.

37. The method of claim 34 wherein each of the plurality of comparison units is further configured for storing a plurality of additional tags.

38. The method of claim 34 wherein the query sequence is comprised of no more than 32 nucleotides of the sequence library.

39. The method of claim 34 wherein the sequence library is comprised of more than one sequence.

40. The method of claim 34 wherein the sequence library is comprised of the genome of a target organism.

41. The method of claim 34 wherein the apparatus is configured to encode the query sequence and the subsequence of the sequence library such that complementation may be performed using bitwise complement.

42. The method of claim 41 wherein the apparatus is configured to encode the query sequence and the subsequence of the sequence library using a binary encoding method where A=00, T=11, C=10, and G=01.

43. A system for locating a query sequence in a sequence library, the system comprising:
a host system comprised of a general purpose computer, the computer being loaded with one or more query sequences and a sequence library;
an apparatus operatively connected to the host system, wherein the apparatus is comprised of a plurality of comparison units and is configured for locating the query sequences in the sequence library;
wherein the host system is responsible for loading tags onto the apparatus, loading the genome onto the apparatus, interrupting results from the apparatus, and saving results from the apparatus.

44. The system of claim 43 wherein the software on the host system also disambiguates the results from the apparatus.

45. The system of claim 43 wherein the host system and the apparatus are operatively connected using a method selected from the group consisting of a CPU bus (e.g. front side bus, HyperTransport, and QuickPath), peripheral component interconnect, peripheral component interconnect express, high speed serial connections, universal serial bus, and AT Attachment.

46. A method of plant breeding comprising:
obtaining a first and a second inbred parental line, the parental lines differing in a plant trait of interest;
crossing the first inbred parental line with the second inbred parental line to create a first filial generation;
crossing the first filial generation with the first inbred parental line to create progeny; growing the progeny, measuring a trait of interest and recording the measurements; collecting DNA from the progeny and sequencing the DNA to produce a number of query sequences;
providing the query sequences and a reference plant genome to an apparatus comprised of a plurality of comparison units configured for locating the query sequences in the reference plant genome;
receiving one or more results from the apparatus indicating the location of the query sequences within the reference plant genome;
analyzing the results from the apparatus to identify a plurality of single nucleotide polymorphisms, possession of which is correlated to desirable phenotypic measurements in the trait of interest;
using the identified plurality of single nucleotide polymorphisms in marker assisted selection to achieve a desired phenotype in a third inbred parental plant line.
